SEQUENCE LISTING (4 sequences)

SEQ ID NO: 1 - DNA, 62 bp, synthetic construct
  source 1..62: mol_type = other DNA
agacaatcaaccatttggggtggacagccttgacctctagacttcggcattttttttttttt

SEQ ID NO: 2 - DNA, 112 bp, synthetic construct
  source 1..112: mol_type = other DNA
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, synthetic construct
  source 1..19: mol_type = other DNA
atgcggggttctcatcatc

SEQ ID NO: 4 - DNA, 20 bp, synthetic construct
  source 1..20: mol_type = other DNA
cgggatccttatcgtcatcg

(19) United States
(12) Patent Application Publication
(10) Pub. No.: US 2025/0154589 A1
(43) Pub. Date: May 15, 2025

(54) FLEXIBLE SUBSTRATES FOR NUCLEIC ACID SYNTHESIS

(71) Applicant: Twist Bioscience Corporation, South San Francisco, CA (US)

(72) Inventor: Bill James Peck, Santa Clara, CA (US)

(73) Assignee: Twist Bioscience Corporation, South San Francisco, CA (US)

(21) Appl. No.: 19/030,426

(22) Filed: Jan. 17, 2025

Related U.S. Application Data
(63) Continuation of application No. 18/050,912, filed on Oct. 28, 2022, now abandoned.
Application No. 18/050,912 is a continuation of application No. 15/272,004, filed on Sep. 21, 2016, now Pat. No. 11,512,347.
(60) Provisional application No. 62/222,020, filed on Sep. 22, 2015.

(51) Int. Cl. C12Q 1/6874; B01J 19/00; C12Q 1/6806

(52) U.S. Cl. CPC C12Q 1/6874; B01J 19/0046; C12Q 1/6806; B01J 2219/00518; B01J 2219/0061; B01J 2219/00632; B01J 2219/00637; B01J 2219/00722

(57) ABSTRACT

Provided herein are compositions, devices, systems and methods for the generation and use of biomolecule-based information for storage. Further described herein are highly efficient methods for long term data storage with 100% accuracy in the retention of information. Additionally, devices described herein for de novo synthesis of oligonucleic acids encoding information related to the original source information may have a flexible material for oligonucleic acids extension.

### CROSS-REFERENCE

This application is a continuation application of U.S. patent application Ser. No. 18/050,912, filed Oct. 28, 2022, which is a continuation of U.S. patent application Ser. No. 15/272,004, filed Sep. 21, 2016, now U.S. Pat. No. 11,512,347, issued on Nov. 29, 2022, which claims the benefit of U.S. Provisional Application No. 62/222,020 filed on Sep. 22, 2015, each which are incorporated herein by reference in their entirety.

### SEQUENCE LISTING

The instant application contains a Sequence Listing which has been submitted electronically in .xml format and is hereby incorporated by reference in its entirety. Said .xml copy, created on Jan. 17, 2025, is named 00415-0010_SequenceListing.xml and is 4,752 bytes in size.

### BACKGROUND

Biomolecule based information storage systems, e.g., DNA-based, have a large storage capacity and stability over time. However, there is a need for scalable, automated, highly accurate and highly efficient systems for generating biomolecules for information storage.

### BRIEF SUMMARY

Provided herein are methods for storing information, comprising: converting an item of information in the form of at least one digital sequence to at least one nucleic acid sequence; providing a flexible structure having a surface; synthesizing a plurality of oligonucleic acids having predetermined sequences collectively encoding for the at least one nucleic acid sequence, wherein the plurality of oligonucleic acids comprises at least about 100,000 oligonucleic acids, and wherein the plurality of oligonucleic acids extends from the surface of the flexible structure; and storing the plurality of oligonucleic acids. Further provided herein are methods wherein synthesizing comprises: depositing nucleosides on the surface at predetermined locations; and moving least a portion of the flexible structure through a bath or emissions from a spray bar. Further provided herein are methods wherein the bath or emissions from a spray bar expose the surface of the structure to an oxidizing reagent or a deblocking reagent. Further provided herein are methods wherein synthesizing further comprises capping the nucleosides deposited on the surface. Further provided herein are methods wherein the nucleosides comprise a nucleoside phosphoramidite. Further provided herein are methods wherein the flexible structure comprises a reel-to-reel tape or a continuous tape. Further provided herein are methods wherein the flexible structure comprises a thermoplastic material. Further provided herein are methods wherein the thermoplastic material comprises a polyaryletherketone. Further provided herein are methods wherein the polyaryletherketone is polyetherketone, polyetherketoneketone, poly(ether ether ketone ketone), polyether ether ketone or polyetherketoneetherketoneketone. Further provided herein are methods wherein the flexible structure comprises nylon, nitrocellulose, polypropylene, polycarbonate, polyethylene, polyurethane, polystyrene, acetal, acrylic, acrylonitrile, butadiene styrene, polyethylene terephthalate, polymethyl methacrylate, polyvinyl chloride, transparent PVC foil, Poly(methyl methacrylate), styrenic polymer, fluorine-containing polymers, polyethersulfone or polyimide. Further provided herein are methods wherein each oligonucleic acid of the plurality of oligonucleic acids comprises from 50 to 500 bases in length. Further provided herein are methods wherein the plurality of oligonucleic acids comprises at least about 10 billion oligonucleic acids. Further provided herein are methods wherein at least about 1.75×10¹³ nucleobases are synthesized within 24 hours. Further provided herein are methods wherein at least about 262.5×10⁹ oligonucleic acids are synthesized within 72 hours. Further provided herein are methods wherein the item of information is text information, audio information or visual information. Further provided herein are methods wherein the nucleosides comprise nucleoside phosphoramidite.

Provided herein are methods for storing information, comprising: converting an item of information in the form of at least one digital sequence to at least one nucleic acid sequence; providing a structure having a surface; synthesizing a plurality of oligonucleic acids having predetermined sequences collectively encoding for the at least one nucleic acid sequence, wherein the plurality of oligonucleic acids comprises at least about 100,000 oligonucleic acids, wherein the plurality of oligonucleic acids extends from the surface of the structure, and wherein synthesizing comprises: cleaning a surface of the structure; depositing nucleosides on the surface at predetermined locations; oxidizing, deblocking, and optionally capping the nucleosides deposited on the surface; wherein the cleaning, oxidizing, deblocking, and capping comprises moving at least a portion of the flexible structure through a bath or emissions from a spray bar; and storing the plurality of oligonucleic acids. Further provided herein are methods wherein the nucleosides comprise nucleoside phosphoramidite.

Provided herein are methods for storing information, comprising: converting an item of information in the form of at least one digital sequence to at least one nucleic acid sequence; synthesizing a plurality of oligonucleic acids having predetermined sequences collectively encoding for the at least one nucleic acid sequence, wherein the plurality of oligonucleic acids comprises at least about 10,000 oligonucleic acids, wherein the plurality of oligonucleic acids collectively encode for a sequence that differs from the predetermined sequences by no more than 1 base in 1000, and wherein each oligonucleic acid of the plurality of oligonucleic acids comprises from 50 to 500 bases in length; and storing the at least about 10,000 oligonucleic acids. Further provided herein are methods wherein the plurality of oligonucleic acids comprises at least about 100,000 oligonucleic acids. Further provided herein are methods wherein the plurality of oligonucleic acids comprises at least about 1,000,000 oligonucleic acids. Further provided herein are methods wherein the plurality of oligonucleic acids comprises at least about 10 billion oligonucleic acids. Further provided herein are methods wherein greater than 90% of the oligonucleic acids encode for a sequence that does not differ from the predetermined sequence. Further provided herein are methods wherein the item of information is text information, audio information or visual information. Further provided herein are methods wherein the structure is rigid or flexible, and wherein the structure comprises a surface, and wherein the plurality of oligonucleic acids extend from the surface. Further provided herein are methods wherein the nucleosides comprise nucleoside phosphoramidite.

Provided herein are methods for storing information, comprising: converting an item of information in the form of at least one digital sequence to at least one nucleic acid sequence; synthesizing a plurality of oligonucleic acids having predetermined sequences collectively encoding for the at least one nucleic acid sequence, wherein the plurality of oligonucleic acids comprises at least about 10,000 oligonucleic acids, wherein each oligonucleic acid of the plurality of oligonucleic acids comprises from 50 to 500 bases in length, and where the plurality of oligonucleic acids extends from the surface of a flexible structure; and storing the plurality of oligonucleic acids. Further provided herein are methods wherein the flexible structure comprises a reel-to-reel tape or a continuous tape. Further provided herein are methods wherein each oligonucleic acid extends from a feature on the surface of the flexible structure, wherein the feature is about 1 μm to about 500 μm in diameter. Further provided herein are methods wherein the feature is about 1 μm to about 50 μm in diameter. Further provided herein are methods wherein the feature is about 10 μm in diameter. Further provided herein are methods wherein the flexible structure comprises a thermoplastic material. Further provided herein are methods wherein the thermoplastic material comprises a polyaryletherketone. Further provided herein are methods wherein the polyaryletherketone is polyetherketone, polyetherketoneketone, poly(ether ether ketone ketone), polyether ether ketone or polyetherketoneetherketoneketone. Further provided herein are methods wherein the flexible structure comprises nylon, nitrocellulose, polypropylene, polycarbonate, polyethylene, polyurethane, polystyrene, acetal, acrylic, acrylonitrile, butadiene styrene, polyethylene terephthalate, polymethyl methacrylate, polyvinyl chloride, transparent PVC foil, Poly(methyl methacrylate), styrenic polymer, fluorine-containing polymers, polyethersulfone or polyimide. Further provided herein are methods wherein the flexible structure has a thickness of less than about 10 mm. Further provided herein are methods wherein each oligonucleic acid is about 200 bases in length. Further provided herein are methods wherein at least about 1.75×10¹³ nucleobases are synthesized within 24 hours. Further provided herein are methods wherein at least about 262.5×10⁹ oligonucleic acids are synthesized within 72 hours. Further provided herein are methods wherein the nucleosides comprise nucleoside phosphoramidite.

Provided herein are methods for storing information, the method comprising: encrypting at least one item of information in the form of at least one digital sequence to at least one nucleic acid sequence; synthesizing a plurality of oligonucleic acids having predetermined sequences collectively encoding for the at least one nucleic acid sequence, wherein the plurality of oligonucleic acids comprises at least about 10,000 oligonucleic acids, and wherein each oligonucleic acid of the plurality of oligonucleic acids comprises from 50 to 500 bases in length; storing the plurality of oligonucleic acids; sequencing the plurality of oligonucleic acids; decrypting the plurality of oligonucleic acids from a nucleic acid sequence to a digital sequence; and assembling the digital sequence to form the at least one digital sequence, wherein the at least one digital sequence is recovered with 100% accuracy. Further provided herein are methods further comprising releasing the plurality of oligonucleic acids. Further provided herein are methods wherein the nucleosides comprise nucleoside phosphoramidite.

Provided herein are devices for information storage, comprising: a flexible structure having a surface; and a plurality of features on the surface, wherein each feature has a width of from about 1 to about 500 μm, and wherein each feature of the plurality of features is coated with a moiety that binds to the surface and comprises a hydroxyl group available for nucleoside coupling. Further provided herein are devices wherein the flexible structure rests in a curved position. Further provided herein are devices wherein the curved position comprises a curve that is greater than 30 degrees. Further provided herein are devices wherein the curved position comprises a curve that is greater than 180 degrees. Further provided herein are devices wherein the flexible structure comprises at least about 1 million features. Further provided herein are devices wherein the flexible structure has a total surface area of less than about 4.5 m². Further provided herein are devices wherein the flexible structure comprises more than 2 billion features per m². Further provided herein are devices wherein the flexible structure comprises a thermoplastic material. Further provided herein are devices wherein the thermoplastic material comprises a polyaryletherketone. Further provided herein are devices wherein the polyaryletherketone is polyetherketone, polyetherketoneketone, poly(ether ether ketone ketone), polyether ether ketone or polyetherketoneetherketoneketone. Further provided herein are devices wherein the flexible structure comprises nylon, nitrocellulose, polypropylene, polycarbonate, polyethylene, polyurethane, polystyrene, acetal, acrylic, acrylonitrile, butadiene styrene, polyethylene terephthalate, polymethyl methacrylate, polyvinyl chloride, transparent PVC foil, Poly(methyl methacrylate), styrenic polymer, fluorine-containing polymers, polyethersulfone or polyimide. Further provided herein are devices wherein the flexible structure has a thickness of less than about 10 mm. Further provided herein are devices wherein each feature is from about 1 um to about 50 um in width. Further provided herein are devices wherein each feature has a diameter of about 10 um. Further provided herein are devices wherein the center of a first feature is about 21 um from the center of a second feature and the first feature and the second feature. Further provided herein are devices wherein the flexible structure comprises a reel-to-reel tape or a continuous tape. Further provided herein are devices wherein each feature comprises a channel.

Provided herein are oligonucleic acid libraries for information storage, comprising a plurality of oligonucleic acids, wherein the plurality of oligonucleic acids comprises at least about 10,000 oligonucleic acids, wherein the plurality of oligonucleic acids collectively encodes for a sequence that differs from an aggregate of predetermined sequences by no more than 1 base in 1000, and wherein each oligonucleic acid of the plurality of oligonucleic acids comprises: a predetermined sequence that, when decrypted, encodes for digital information; and from 50 to 500 bases in length. Further provided herein are libraries wherein the plurality of oligonucleic acids comprises at least about 100,000 oligonucleic acids. Further provided herein are libraries wherein the plurality of oligonucleic acids comprises at least about 10 billion oligonucleic acids. Further provided herein are libraries wherein each oligonucleic acid of the plurality of oligonucleic acids is attached to a surface of a structure by a tether. Further provided herein are libraries wherein the tether comprises a cleavable region having at least one nucleotide chemically modified to detach from the oligonucleic acid in the presence of a cleaving reagent. Further provided herein are libraries wherein the tether comprises from about 10 to about 50 bases. Further provided herein are libraries wherein greater than 90% of the oligonucleic acids encode for a sequence that does not differ from the predetermined sequences. Further provided herein are libraries wherein the digital information encodes for text, audio or visual information. Further provided herein are libraries wherein the library is synthesized in less than 3 days. Further provided herein are libraries wherein the library is synthesized in less than 24 hours.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary workflow for nucleic acid-based data storage.

FIG. 2A illustrates an exemplary continuous workflow having a tape, rolling units and a material deposition unit.

FIG. 2B illustrates an exemplary zoom in view of the tap in FIG. 2A, showing discrete loci for oligonucleic acid extension.

FIG. 3 illustrates a portion of surface having features that support oligonucleic acid synthesis.

FIG. 4 illustrates an example of a computer system.

FIG. 5 is a block diagram illustrating an architecture of a computer system.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space.

### DETAILED DESCRIPTION

There is a need for larger capacity storage systems as the amount of information generated and stored is increasing exponentially. Traditional storage media have a limited capacity and require specialized technology that changes with time, requiring constant transfer of data to new media, often at a great expense. A biomolecule such as a DNA molecule provides a suitable host for information storage in-part due to its stability over time and capacity for four bit information coding, as opposed to traditional binary information coding. Thus, large amounts of data are encoded in the DNA in a relatively smaller amount of physical space than used by commercially available information storage devices.

### Definitions

Unless defined otherwise, all technical and scientific terms used herein have the same meaning as is commonly understood by one of ordinary skill in the art to which these inventions belong.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Nucleic Acid Based Information Storage

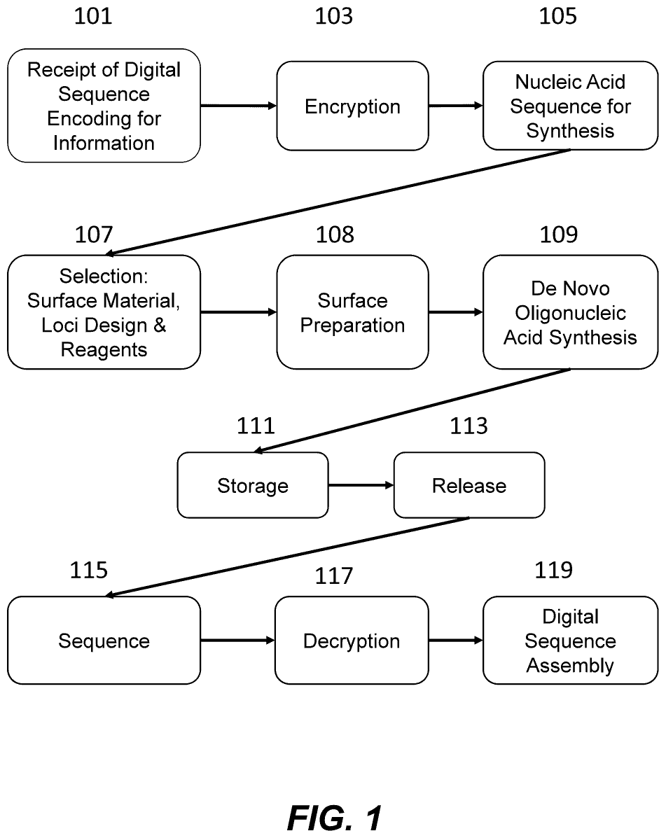

Provided herein are devices, compositions, systems and methods for nucleic acid-based information (data) storage. An exemplary workflow is provided in FIG. 1. In a first step, a digital sequence encoding an item of information (i.e., digital information in a binary code for processing by a computer) is received 101. An encryption 103 scheme is applied to convert the digital sequence from a binary code to a nucleic acid sequence 105. A surface material for nucleic acid extension, a design for loci for nucleic acid extension (aka, arrangement spots), and reagents for nucleic acid synthesis are selected 107. The surface of a structure is prepared for nucleic acid synthesis 108. De novo oligonucleic acid synthesis is performed 109. The synthesized oligonucleic acids are stored 111 and available for subsequent release 113, in whole or in part. Once released, the oligonucleic acids, in whole or in part, are sequenced 115, subject to decryption 117 to convert nucleic sequence back to digital sequence. The digital sequence is then assembled 119 to obtain an alignment encoding for the original item of information.

### Information Storage

Provided herein are methods and systems for storing information encoded by biomolecules on a substrate. In some instances, the information is digital data. In some instances, the biomolecules comprise DNA. In some cases, the biomolecules comprise oligonucleic acids. In some instances, methods are provided for the synthesis of the oligonucleic acids onto the substrate. In some instances, the synthesized oligonucleic acids are positioned on the substrate at a high density to encode large and complex amounts of data in a small footprint. Exemplary substrates are flexible, allowing for the manipulation of the substrate during synthesis, storage, and/or data extraction. In some instances, the flexible substrates are configured for rolling onto a reel for long term storage.

To store data in a sequence of DNA, the information is converted from the 1s and 0s of binary code into the code of A, T, G, and C bases of DNA. In some instances, items of information are first encoded in a digital information form. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profiles sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, .gif, .psd, .bmp, .tiff, .png, and .mpeg. In some instances, the binary code of digital sequence is converted into a biomolecule-based (e.g., DNA-based) sequence while preserving the information that the code represents. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1 PB), 1 exabyte, 1 zettabyte, 1 yottabyte, 1 xenottabyte or more. This converted code (digital binary code to a biomolecule code) is referred to herein as “predetermined” sequence with respect to the deposit of a biomolecule disclosed herein on a surface disclosed herein.

A predetermined sequence comprising the converted DNA code is synthesized into one or a plurality of oligonucleic acids that are supported on a structure (aka substrate) for data storage. In some instances, the oligonucleic acids are synthesized on the substrate using an oligonucleic acid synthesizer device that releases nucleic acid synthesis reagents in a step wise fashion such that that multiple oligonucleic acids extend, in parallel, one residue at a time from the surface of the substrate. Each oligonucleic acid is positioned on distinct regions, or features, of the substrate. In many cases, these regions are positioned in addressable locations of the substrate. In some instances, two or more of the oligonucleic acids on a substrate have sequences that differ. In some instances, two or more of the oligonucleic acids on a substrate have sequences that are the same.

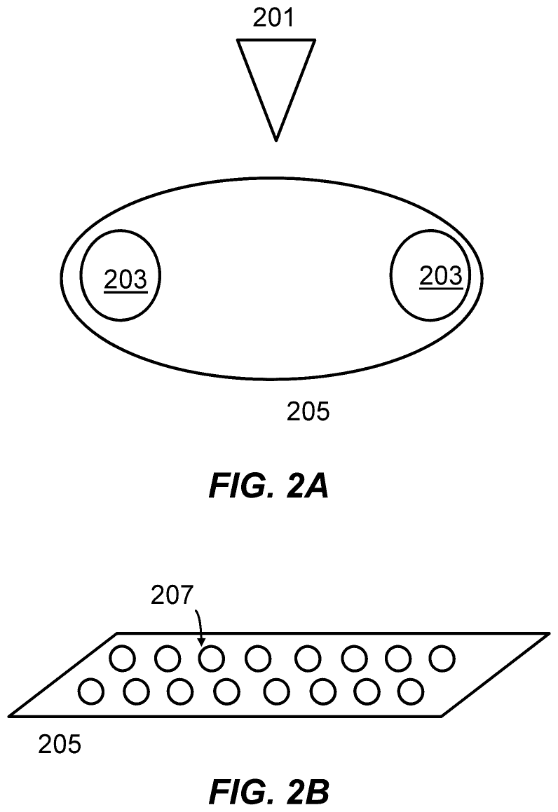

A structure described herein for oligonucleic acid extension during synthesis may be a rigid or flexible material. An exemplary process workflow for de novo synthesis of an oligonucleic acid on a substrate using an oligonucleic acid synthesizer is shown in FIG. 2A and FIG. 2B. In the illustration, an oligonucleic acid synthesis material deposition unit 201 releases reagents onto a flexible structure 205 (the substrate) comprising a surface, wherein the surface comprises a plurality of features 207 (or “loci”) for nucleic acid extension. In the continuous belt arrangement, the flexible 205 structure is wrapped around rollers 203.

In some instances, a substrate that supports the synthesis and storage of oligonucleic acids encoding information comprises a flexible material. In some cases, the flexible material is in the form of a tape. In some cases, substrates having flexible materials are used in a reel-to-reel tape, where a first end of the substrate is attached (reversibly or irreversibly) to a first reel and a second end of the substrate is attached (reversibly or irreversibly) to a second reel. In this manner, the body of the substrate is be wrapped around the first reel, the second reel, or both. The reels of the system are rotatable so that the substrate is transferred between the reels while in use. During an oligonucleic acid synthesis reaction performed on a substrate of a reel-to-reel tape system, sections of the substrate pass through various stages of the synthesis reaction in a production assembly line manner. As an example, a portion of the substrate passes through a stage at which a nucleobase is attached to the substrate during a nucleic acid synthesis reaction. In another example, a portion of the substrate passes through a wash stage of a nucleic acid synthesis reaction. In some cases, one portion of a substrate is positioned at a different stage of a nucleic acid synthesis reaction than another portion of the substrate.

In some instances, a flexible material described herein for oligonucleic acid synthesis comprises continuous tape. In some instances, a substrate for the synthesis and/or storage of oligonucleic acids comprises a flexible material that is rotatable around a rotating drum in a continuous conveyor belt configuration or a “continuous tape system.” In an exemplary continuous tape system, oligonucleic acid synthesis steps are partitioned into zones and regions of the substrate are conveyed continuously through each of the zones. As an example, an oligonucleic acid synthesis reaction proceeds by conveying a flexible substrate from a deposition zone where droplets comprising oligonucleic acid building blocks are deposited and coupled onto the conveyed substrate surface, to one or more processing zones (e.g., capping, oxidation, washing, drying) in a continuous cycle, extending the synthesized oligonucleic acids by a single base in each cycle. In some instances, continuous conveyance of a substrate through an oligonucleic acid synthesis reaction proceeds with more efficiency as compared to an oligonucleic acid synthesis reaction that occurs in distinct steps because multiple chemistries are performed on different regions of the substrate at the same time.

In another exemplary continuous tape system, the entire continuous tape is exposed to a single step in a reaction as the tape proceeds in a rotatable fashion. After each portion of the surface of the tape is exposed to reaction step in a single pass, the next step of the reaction occurs. As an example, an oligonucleic acid synthesis reaction proceeds by conveying the tape through a section of a device that releases an oxidizing reagent. After the entire tape is receives nucleoside monomer deposition, the tape is then exposed to a washing step, followed by a rounds of oxidation, washing, deblocking, washing, capping, washing and then repeating, resulting in extending the synthesized oligonucleic acids by a single base in each cycle.

The DNA code of synthesized and stored oligonucleic acids is read either directly on the substrate, or after extraction from the substrate, by using any suitable sequencing technology. In some cases, the DNA sequence is read on the substrate or within a feature of a substrate. In some cases, the oligonucleic acids stored on the substrate are extracted is optionally assembled into longer nucleic acids and then sequenced.

Provided here are systems and methods configured to synthesize a high density of oligonucleic acids on a substrate in a short amount of time. In some cases, the substrate is a flexible substrate. In some instances, at least about 10¹⁰, 10¹¹, 10¹², 10¹³, 10¹⁴, or 10¹⁵ bases are synthesized in one day. In some instances, at least about 10×10⁸, 10×10⁹, 10×10¹⁰, 10×10¹¹, or 10×10¹² oligonucleic acids are synthesized in one day. In some cases, each oligonucleic acid synthesized comprises at least about 20, 50, 100, 200, 300, 400 or 500 nucleobases. In an example, at least 10×10⁹, 200 base oligonucleic acids are synthesized within 3 days. In some cases, these bases are synthesized with a total average error rate of less than about 1 in 100; 200; 300; 400; 500; 1000; 2000; 5000; 10000; 15000; 20000 bases.

Oligonucleic acids synthesized and stored on the substrates described herein encode data that can be interpreted by reading the sequence of the synthesized oligonucleic acids and converting the sequence into binary code (“decrypting”) readable by a computer. In a further aspect, provided is a detection system comprising a device capable of sequencing stored oligonucleic acids, either directly on the substrate and/or after removal from the substrate. In cases where the substrate is a reel-to-reel tape of flexible material, the detection system comprises a device for holding and advancing the substrate through a detection location and a detector disposed proximate the detection location for detecting a signal originated from a section of the tape when the section is at the detection location. In some instances, the signal is indicative of a presence of an oligonucleic acid. In some instances, the signal is indicative of a sequence of an oligonucleic acid. In another aspect, described herein are detection methods for detecting and reading a biomolecule stored on a substrate. In cases where the substrate is a flexible material on a reel-to-reel tape, the method comprises sequentially advancing through a fixed position the substrate for sequential detection and reading of bound biomolecules. In some instances, information encoded within oligonucleic acids on a continuous tape is read by a computer as the tape is conveyed continuously through a detector operably connected to the computer. In some instances, a detection system comprises a computer system comprising an oligonucleic acid sequencing device, a database for storage and retrieval of data relating to oligonucleic acid sequence, software for converting DNA code of an oligonucleic acid sequence to binary code, a computer for reading the binary code, or any combination thereof.

In a further aspect of the disclosure, provided is a cassette that comprises a housing and a tape, wherein the tape is a flexible substrate comprising a plurality of attached biomolecules. The tape is housed in the housing such that the tape is advanceable along a path from a first end to a second end of the tape.

### Structures

Provided herein are structures (also referred to as substrates) comprising a plurality of features, wherein biomolecules are attached directly or indirectly to a surface of the structure. In many cases, the biomolecules comprise nucleic acid sequences that are synthesized on features of the substrate. In some instances, the features are closely spaced so that a small area of the structure encodes a high density of data. For example, the distance between the centers of two features is from about 1 um to about 200 um, from about 1 um to about 100 um, from about 1 um to about 50 um, from about 1 um to about 25 um, from about 10 um to about 50 um, or from about 10 um to about 25. In some cases, the distance between two features is less than about 100 um, 50 um, 40 um, 30 um, 20 um or 10 um. The size of each feature may range from about 0.1 um to about 100 um, from about 1 um to about 100 um, from about 1 um to about 50 um, or from about 0.1 um to about 100 um. In some cases, each feature is less than about 100 um, 50 um, 20 um, 10 um, or 5 um in diameter. In some instances, each square meter of a structure allows for at least about 10⁷, 10⁸, 10⁹, 10¹⁰, 10¹¹ features, where each feature supports one oligonucleic acid. In some cases, the oligonucleic acids have lengths up to about 100, 200, 300, 400, 500 or more bases. In some instances, 10⁹ oligonucleic acids are supported on less than about 6, 5, 4, 3, 2 or 1 m² of surface of the structure.

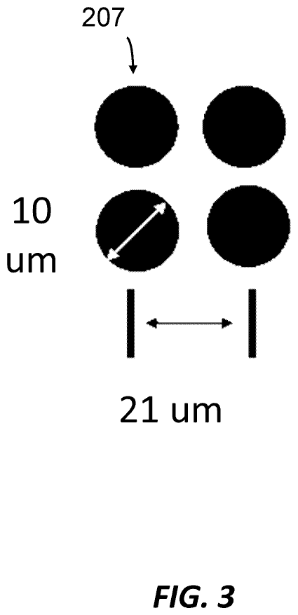

To illustrate exemplary dimensions of a structure described herein, reference is made to FIG. 3. Reference to this figure is for example purposes only, and the numbers, dimensions and configuration of features described are not limiting. The region of the surface of a the structure shown in FIG. 3 illustrates four features of 10 um in diameter, at a center-to-center distance of 21 um. The features of FIG. 3 are arranged in rows forming a square shape, however, it is intended that the features may be arranged in any configuration, for example, without rows or in a circular or staggered shape.

### Flexible Structures

Provided herein are flexible structures that allow for manipulation during biomolecule attachment, storage and/or reading. The term “flexible” is used herein to refer to a structure that is capable of being bent, folded or similarly manipulated without breakage. In some instances, a flexible structures is bent 180 degrees around a roller. In some instances, a flexible structure is bent about 30 to about 330 degrees around a roller. In some instances, a flexible structure is bent up to about 360 degrees around a roller. In some cases, the roller is less than about 10 cm, 5 cm, 3 cm, 2 cm or 1 cm in radius. In some instances, the structures is bent and straightened repeatedly in either direction at least 100 times without failure (for example, cracking) or deformation at 20° C. In some instances, a structure comprises rigid materials. In some cases, a structure has a thickness that is amenable to rolling. In some cases, the thickness of the structure is less than about 500 mm, 100 mm, 50 mm, 10 mm, or 1 mm. In some cases, the thickness of the structure is less than about 1 mm, 0.5 mm, 0.1 mm, 0.05, 0.01, or thinner.

Exemplary flexible materials described herein include, without limitation, nylon (unmodified nylon, modified nylon, clear nylon), nitrocellulose, polypropylene, polycarbonate, polyethylene, polyurethane, polystyrene, acetal, acrylic, acrylonitrile, butadiene styrene (ABS), polyester films such as polyethylene terephthalate, polymethyl methacrylate or other acrylics, polyvinyl chloride or other vinyl resin, transparent PVC foil, transparent foil for printers, Poly(methyl methacrylate) (PMMA), methacrylate copolymers, styrenic polymers, high refractive index polymers, fluorine-containing polymers, polyethersulfone, polyimides containing an alicyclic structure, rubber, fabric, metal foils, and any combination thereof. Various plasticizers and modifiers may be used with polymeric materials to achieve selected flexibility characteristics.

In some instances, the structure comprises a plastic material. In some instances, the structure comprises a thermoplastic material. Non-limiting examples of thermoplastic materials include acrylic, acrylonitrile butadiene styrene, nylon, polylactic acid, polybenzimidazole, polycarbonate, polyether sulfone, polyetherether ketone, polyetherimide, polyethylene, polyphenylene oxide, polyphenylene sulfide, polypropylene, polystyrene, polyvinyl chloride, and polytetrafluoroethylene. In some instances, the structure comprises a thermoplastic material in the polyaryletherketone (PEAK) family. Non-limiting examples of PEAK thermoplastics include polyetherketone (PEK), polyetherketoneketone (PEKK), poly(ether ether ketone ketone) (PEEKK), polyether ether ketone (PEEK), and polyetherketoneetherketoneketone (PEKEKK). In some instances, the structure comprises a thermoplastic material compatible with toluene. In some cases, the flexibility of the plastic material is increased by the addition of a plasticizer. An example of a plasticizer is an ester-based plasticizer, such as phthalate. Phthalate plasticizers include bis(2-ethylhexyl) phthalate (DEHP), diisononly phthalate (DINP), di-n-butyl phthalate (DnBP, DBP), butyl benzyl phthalate (BBzP), diisodecyl phthalate (DIDP), dioctyl phthalate (DOP, DnOP), diisooctyl phthalate (DIOP), diethyl phthalate (DEP), diisobutyl phthalate (DIBP), and di-n-hexyl phthalate. In some instances, modification of the thermoplastic polymer through copolymerization or through the addition of non-reactive side chains to monomers before polymerization also increases flexibility.

In some instances, the structure comprises a fluoroelastomer. Materials having about 80% fluoroelastomers are designated as FKMs. Fluoroelastomers include perfluoro-elastomers (FFKMs) and tetrafluoroethylene/propylene rubbers (FEPM). Fluoroelastomers have five known types. Type 1 FKMs are composed of vinylidene fluoride (VDF) and hexafluoropropylene (HFP) and their fluorine content typically is around 66% by weight. Type 2 FKMs are composed of VDF, HFP, and tetrafluoroethylene (TFE) and typically have between about 68% and 69% fluorine. Type 3 FKMs are composed of VDF, TFE, and perfluoromethylvinylether (PMVE) and typically have between about 62% and 68% fluorine. Type 4 FKMs are composed of propylene, TFE, and VDF and typically have about 67% fluorine. Type 5 FKMs are composed of VDF, HFP, TFE, PMVE, and ethylene.

In some instances, a structure disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the structure comprises magnetic reel-to-reel tape or a magnetic belt. In some cases, the structure comprises a flexible printed circuit board.

In some instances, a substrate material disclosed herein is transparent to visible and/or UV light. In some instances, substrate materials are sufficiently conductive to form uniform electric fields across all or a portion of a substrate. In some cases, the substrate is heat conductive or insulated. In some cases, the materials are chemical resistant and heat resistant to support a chemical reaction such as an oligonucleic acid synthesis reaction. In some instances, the substrate is magnetic. In some instances, the substrate comprises a metal or a metal alloy.

In some instances, a surface comprises a rigid material. A rigid material includes, without limitation, glass; fused silica; silicon such as silicon dioxide or silicon nitride; metals such as gold or platinum; plastics such as polytetrafluoroethylene, polypropylene, polystyrene, polycarbonate, and any combination thereof.

In some instances, a substrate material disclosed herein comprises a flat region. In some instances, the substrate comprises embedded pores, which are a series of individual reaction sections that capture released oligonucleic acids, facilitating direct sequencing of the oligonucleic acids within the pores of the substrate. In some cases, a substrate material disclosed herein comprises pores. In some cases the pores are coated with a functionalizing agent disclosed herein where the agent couples nucleoside base to the surface of a substrate. In some cases, the pores comprise microchannels. In some cases, a single pore comprises at least 2 microchannels. In some cases, a single pore contains about 2 to about 200, about 100 to about 150 microchannels. In some cases, the micropores are coated with a functionalizing agent disclosed herein where the agent couples nucleoside base to the surface of a substrate. In some cases, a substrate material disclosed herein comprises wells. In some cases the wells are coated with a functionalizing agent disclosed herein where the agent couples nucleoside base to the surface of a substrate. In some cases, deposition of a monomeric oligonucleotide in a manner described herein is into a pore, microchannel or well on the surface of a substrate. In some cases, reading of an oligonucleic acid synthesized by methods disclosed herein occurs within a pore, microchannel, or well on the surface of the substrate.

In some instances, the substrate comprises an alignment structure or printed alignment element, such as a fiducial marking. In some instances, the substrate comprises a detectable marker attached to a section of the substrate for identifying that section. In some cases, the substrate comprises one or more regions for annotation. In some cases, the substrate is labeled.

In some cases, a substrate disclosed herein comprises one or more identifiers. In some instances, each identifier is associated with each biomolecule on a substrate, or a group of biomolecules on a substrate, by having a fixed location on the substrate in relation to a bar code from which relative location the identity of each biomolecule or group of biomolecules is determined. In one aspect, an identifier provides a means to identify biomolecule information. In some cases the biomolecule is an oligonucleic acid and the information is the sequence identity. In some cases, the information is stored in a database.

### Surface Modification

In some instances, to support the immobilization of a biomolecule on a substrate for de novo synthesis of nucleic acids, the surface of the structure comprises a material and/or is coated with a material that facilitates a coupling reaction with the biomolecule for attachment. In various instances, to prepare a substrate for biomolecule immobilization, surface modifications are employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. In some cases, a substrate is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

In some instances, the surface of the substrate is modified to comprise one or more actively functionalized surfaces configured to bind to both the surface of the substrate and a biomolecule, thereby supporting a coupling reaction to the surface. In some cases, the surface is also functionalized with a passive material that does not efficiently bind the biomolecule, thereby preventing biomolecule attachment at sites where the passive functionalization agent is bound. In some cases, the surface comprises an active layer only defining distinct features for biomolecule support. In some cases, the surface is not coated.

In some instances, the substrate surface is contacting with a mixture of functionalization groups which are in any different ratio. In some instances, a mixture comprises at least 2, 3, 4, 5 or more different types of functionalization agents. In some cases, the ratio of the at least two types of surface functionalization agents in a mixture is about 1:1, 1:2, 1:5, 1:10, 2:10, 3:10, 4:10, 5:10, 6:10, 7:10, 8:10, 9:10, or any other ratio to achieve a desired surface representation of two groups. In some instances, desired surface tensions, wettabilities, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some instances, the mixture of functionalization reagents comprises one or more reagents that bind to a biomolecule and one or more reagents that do not bind to a biomolecule. Therefore, modulation of the reagents allows for the control of the amount of biomolecule binding that occurs at a distinct area of functionalization.

In some instances, a method for substrate functionalization comprises deposition of a silane molecule onto a surface of a substrate. In some instances, the silane molecule is deposited on a high energy surface of the substrate. In some instances the high surface energy region includes a passive functionalization reagent. The silane group binds to the surface, while the rest of the molecule provides a distance from the surface and a free hydroxyl group at the end to which a biomolecule attaches. In some instances, the silane is an organofunctional alkoxysilane molecule. Non-limiting examples of organofunctional alkoxysilane molecules include dimethylchloro-octodecyl-silane, methyldichloro-octodecyl-silane, trichloro-octodecyl-silane, and trimethyl-octodecyl-silane, triethyl-octodecyl-silane. In some instances, the silane is an amino silane. Examples of amino silanes include, without limitation, 11-acetoxyundecyltriethoxysilane, n-decyltriethoxysilane, (3-aminopropyl) trimethoxysilane, (3-aminopropyl)triethoxysilane, glycidyloxypropyl/trimethoxysilane and N-(3-triethoxysilylpropyl)-4-hydroxybutyramide. In some instances, the silane comprises 11-acetoxyundecyltriethoxysilane, n-decyltriethoxysilane, (3-aminopropyl) trimethoxysilane, (3-aminopropyl)triethoxysilane, glycidyloxypropyl/trimethoxysilane, N-(3-triethoxysilylpropyl)-4-hydroxybutyramide, or any combination thereof. In some cases, an active functionalization agent comprises 11-acetoxyundecyltriethoxysilane. In some cases, an active functionalization agent comprises n-decyltriethoxysilane. In some cases, an active functionalization agent comprises glycidyloxypropyltriethoxysilane (GOPS). In some instances, the silane is a fluorosilane. In some instances, the silane is a hydrocarbon silane. In some cases, the silane is 3-iodo-propyltrimethoxysilane. In some cases, the silane is octylchlorosilane.

In some instances, silanization is performed on a surface through self-assembly with organofunctional alkoxysilane molecules. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxysilane or 3-mercaptopropyl-methyl-dimethoxysilane), bicyclohepthenyl-trichlorosilane, butyl-aldehydr-trimethoxysilane, or dimeric secondary aminoalkyl siloxanes. Exemplary hydroxyalkyl siloxanes include allyl trichlorochlorosilane turning into 3-hydroxypropyl, or 7-oct-1-enyl trichlorochlorosilane turning into 8-hydroxyoctyl. The diol (dihydroxyalkyl) siloxanes include glycidyl trimethoxysilane-derived (2,3-dihydroxypropyloxy) propyl (GOPS). The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). In some cases, the dimeric secondary aminoalkyl siloxanes is bis(3-trimethoxysilylpropyl) amine turning into bis(silyloxylpropyl)amine.

In some instances, active functionalization areas comprise one or more different species of silanes, for example, 1, 2, 3, 4, 5, 6, 7, 8, 9, 10 or more silanes. In some cases, one of the one or more silanes is present in the functionalization composition in an amount greater than another silane. For example, a mixed silane solution having two silanes comprises a 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 75:25, 70:30, 65:35, 60:40, 55:45 ratio of one silane to another silane. In some instances, an active functionalization agent comprises 11-acetoxyundecyltriethoxysilane and n-decyltriethoxysilane. In some instances, an active functionalization agent comprises 11-acetoxyundecyltriethoxysilane and n-decyltriethoxysilane in a ratio from about 20:80 to about 1:99, or about 10:90 to about 2:98, or about 5:95.

### Synthesis on a Substrate

The substrates described herein may comprise a plurality of features that allow for the attachment and synthesis of oligonucleic acids to the surface. In some instances, droplets comprising oligonucleic acid synthesis reagents are released from oligonucleic acid synthesis material deposition unit to the substrate in a stepwise manner from a deposition device having a piezo ceramic material and electrodes to convert electrical signals into a mechanical signal for releasing the droplets. The droplets are release to specific locations on the surface of the substrate one nucleobase at a time to generate a plurality of synthesized oligonucleic acids having predetermined sequences that encode data. In some cases, the synthesized oligonucleic acids are stored on the substrate. In some cases, oligonucleic acids are cleaved from the surface. Cleavage includes gas cleavage with such gases as ammonia or methylamine.

Provided herein are structures that may comprise a surface that supports the synthesis of a plurality of oligonucleic acids having different predetermined sequences at addressable locations on a common support. In some instances, a device provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical oligonucleic acids. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more oligonucleic acids encoding for distinct sequences. In some instances, the device provides support for the synthesis of more than 1 million, 1 billion, 10 billion or more oligonucleic acids. In some instances, at least a portion of the oligonucleic acids have an identical sequence or are configured to be synthesized with an identical sequence.

Provided herein are methods and devices for manufacture and growth of oligonucleic acids about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the oligonucleic acid formed is about 5, 10, 20, 30, 40, 50, 60, 70, 80, 90, 100, 125, 150, 175, 200, or 225 bases in length. An oligonucleic acid may be at least 5, 10, 20, 30, 40, 50, 60, 70, 80, 90, or 100 bases in length. An oligonucleic acid may be from 10 to 225 bases in length, from 12 to 100 bases in length, from 20 to 150 bases in length, from 20 to 130 bases in length, from 25 to 1000 bases in length, from 75 to 500 bases in length, from 30 to 100 bases in length, or from 50 to 500 bases in length.

In some instances, oligonucleic acids are synthesized on distinct loci of a substrate, wherein each locus supports the synthesis of a population of oligonucleic acids. In some instances, each locus supports the synthesis of a population of oligonucleic acids having a different sequence than a population of oligonucleic acids grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct oligonucleic acids synthesized on a device may be dependent on the number of distinct loci available in the substrate. In some instances, the density of loci (or feature) within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 um to about 100 um. In some instances, the distance from two centers of adjacent loci is greater than about 10 um, 20 um, 30 um, 40 um, 50 um, 60 um, 70 um, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 um, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um or 100 um. In some instances, the each locus has a width of about 0.5 um to 100 um, about 0.5 um to 50 um, about 10 um to 75 um, about 0.5 um to 50 um, or about 1 um to about 500 um.

In some cases, synthesized oligonucleic acids disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

A suitable method for oligonucleic acid synthesis on a substrate of this disclosure is a phosphoramidite method comprising the controlled addition of a phosphoramidite building block, i.e. nucleoside phosphoramidite, to a growing oligonucleic acid chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, an oligonucleic acid synthesis method used herein comprises 1, 2, 3 or more sequential coupling steps. Prior to coupling, in many cases, the nucleoside bound to the substrate is de-protected by removal of a protecting group, where the protecting group functions to prevent polymerization. A common protecting group is 4,4′-dimethoxytrityl (DMT).

Following coupling, phosphoramidite oligonucleic acid synthesis methods optionally comprise a capping step. In a capping step, the growing oligonucleic acid is treated with a capping agent. A capping step generally serves to block unreacted substrate-bound 5′-OH groups after coupling from further chain elongation, preventing the formation of oligonucleic acids with internal base deletions. Further, phosphoramidites activated with 1H-tetrazole often react, to a small extent, with the O6 position of guanosine. Without being bound by theory, upon oxidation with I₂/water, this side product, possibly via O6-N7 migration, undergoes depurination. The apurinic sites can end up being cleaved in the course of the final deprotection of the oligonucleotide thus reducing the yield of the full-length product. The O6 modifications may be removed by treatment with the capping reagent prior to oxidation with I₂/water. In some instances, inclusion of a capping step during oligonucleic acid synthesis decreases the error rate as compared to synthesis without capping. As an example, the capping step comprises treating the substrate-bound oligonucleic acid with a mixture of acetic anhydride and 1-methylimidazole. Following a capping step, the substrate is optionally washed.

In some instances, following addition of a nucleoside phosphoramidite, and optionally after capping and one or more wash steps, the substrate bound growing nucleic acid is oxidized. The oxidation step comprises oxidizing the phosphite triester into a tetracoordinated phosphate triester, a protected precursor of the naturally occurring phosphate diester internucleoside linkage. In some cases, oxidation of the growing oligonucleic acid is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing oligonucleic acid is optionally washed. In some instances, the step of oxidation is substituted with a sulfurization step to obtain oligonucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, a protected 5′ end of the substrate bound growing oligonucleic acid must be removed so that the primary hydroxyl group can react with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound oligonucleotide and thus reduces the yield of the desired full-length product. Methods and compositions described herein provide for controlled deblocking conditions limiting undesired depurination reactions. In some cases, the substrate bound oligonucleic acid is washed after deblocking. In some cases, efficient washing after deblocking contributes to synthesized oligonucleic acids having a low error rate.

Methods for the synthesis of oligonucleic acids on the substrates described herein typically involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some cases, one or more wash steps precede or follow one or all of the steps.

In some instances, oligonucleic acids are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

In some instances, the surface of the substrate that provides support for oligonucleic acid synthesis is chemically modified to allow for the synthesized oligonucleic acid chain to be cleaved from the surface. In some cases, the oligonucleic acid chain is cleaved at the same time as the oligonucleic acid is deprotected. In some cases, the oligonucleic acid chain is cleaved after the oligonucleic acid is deprotected. In an exemplary scheme, a trialkoxysilyl amine such as (CH3CH2O)3Si—(CH2)2-NH2 is reacted with surface SiOH groups of a substrate, followed by reaction with succinic anhydride with the amine to create an amide linkage and a free OH on which the nucleic acid chain growth is supported.

Oligonucleic acids synthesized using the methods and substrates described herein are optionally released from the surface from which they are synthesized. In some cases, oligonucleic acids are cleaved from the surface after synthesis. In some cases, oligonucleic acids are cleaved from the surface after storage. Cleavage includes gas cleavage with ammonia or methylamine. In some instances, the application of ammonia gas simultaneous deprotects phosphates groups protected during the synthesis steps, i.e. removal of electron-withdrawing cyano group. In some instances, once released from the surface, oligonucleic acids are assembled into larger nucleic acids that are sequenced and decoded to extract stored information. In some cases, wherein the oligonucleic acids stored on the substrate are to be removed, each sequence fragment comprises an index that provides instructions for how to assemble it with other sequences stored with it.

In some instances, synthesized oligonucleic acids are designed to collectively span a large region of a predetermined sequence that encodes for information. In some instances, larger oligonucleic acids are generated through ligation reactions to join the synthesized oligonucleic acids. One example of a ligation reaction is polymerase chain assembly (PCA). In some cases, at least of a portion of the oligonucleic acids are designed to include an appended region that is a substrate for universal primer binding. For PCA reactions, the presynthesized oligonucleic acids include overlaps with each other (e.g., 4, 20, 40 or more bases with overlapping sequence). During the polymerase cycles, the oligonucleic acids anneal to complementary fragments and then are filled in by polymerase. Each cycle thus increases the length of various fragments randomly depending on which oligonucleic acids find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences is amplified in a polymerase chain reaction (PCR) which includes modified primers that hybridize to the adapter sequences. In some cases, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

In some instances, error correction is performed on synthesized oligonucleic acids and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some instances, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

### Error Rate

In some instances, these error rates are for at least 50%, 60%, 70%, 80%, 90%, 95%, 98%, 99%, 99.5%, or more of the oligonucleic acids synthesized. In some instances, these at least 90%, 95%, 98%, 99%, 99.5%, or more of the oligonucleic acids synthesized do not differ from a predetermined sequence for which they encode. In some instances, the error rate for synthesized oligonucleic acids on a substrate using the methods and systems described herein is less than about 1 in 200. In some instances, the error rate for synthesized oligonucleic acids on a substrate using the methods and systems described herein is less than about 1 in 1,000. In some instances, the error rate for synthesized oligonucleic acids on a substrate using the methods and systems described herein is less than about 1 in 2,000. In some instances, the error rate for synthesized oligonucleic acids on a substrate using the methods and systems described herein is less than about 1 in 3,000. In some instances, the error rate for synthesized oligonucleic acids on a substrate using the methods and systems described herein is less than about 1 in 5,000. Individual types of error rates include mismatches, deletions, insertions, and/or substitutions for the oligonucleic acids synthesized on the substrate. The term “error rate” refers to a comparison of the collective amount of synthesized oligonucleic acid to an aggregate of predetermined oligonucleic acid sequences.

Average error rates for oligonucleic acids synthesized within a library using the systems and methods provided may be less than 1 in 1000, less than 1 in 1250, less than 1 in 1500, less than 1 in 2000, less than 1 in 3000 or less often. In some instances, average error rates for oligonucleic acids synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for oligonucleic acids synthesized within a library using the systems and methods provided are less than 1/1000.

In some instances, aggregate error rates for oligonucleic acids synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for oligonucleic acids synthesized within a library using the systems and methods provided are less than 1/500, 1/600, 1/700, 1/800, 1/900, or 1/1000. In some instances, aggregate error rates for oligonucleic acids synthesized within a library using the systems and methods provided are less than 1/1000.

In some instances, an error correction enzyme may be used for oligonucleic acids synthesized within a library using the systems and methods provided can use. In some instances, aggregate error rates for oligonucleic acids with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for oligonucleic acids synthesized within a library using the systems and methods provided can be less than 1/500, 1/600, 1/700, 1/800, 1/900, or 1/1000. In some instances, aggregate error rates with error correction for oligonucleic acids synthesized within a library using the systems and methods provided can be less than 1/1000.

Libraries disclosed herein may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic oligonucleotide libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the oligonucleotides in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the oligonucleotides in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any oligonucleotides related with more than 95%, 96%, 97%, 98%, 99%, 99.5%, 99.6%, 99.7%, 99.8%, 99.9% or more similarity or identity have the same sequence. In some instances, the error rate related to a specified locus on an oligonucleotide is optimized. Thus, a given locus or a plurality of selected loci of one or more oligonucleotides as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more oligonucleotides.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

### Devices

Provided herein are systems and devices for the deposition and storage of biomolecules on a substrate. In some instances, the biomolecules are oligonucleic acids that store encoded information in their sequences. In some instances, the system comprises a substrate to support biomolecule attachment and/or a device for application of a biomolecule to the surface of the substrate. In an example, the device for biomolecule application is an oligonucleic acid synthesizer. In some instances, the system comprises a device for treating the substrate with a fluid, for example, a flow cell. In some instances, the system comprises a device for moving the substrate between the application device and the treatment device. For instances where the substrate is a reel-to-reel tape, the system may comprise two or more reels that allow for access of different portions of the substrate to the application and optional treatment device at different times.

In some instances, a flexible substrate comprising thermoplastic material is coated with nucleoside coupling reagent. The coating is patterned into features such that each feature has diameter of about 10 um, with a center-to-center distance between two adjacent features of about 21 μm. In this case, the feature size is sufficient to accommodate a sessile drop volume of 0.2 pl during an oligonucleic acid synthesis deposition step. In some cases, the feature density is about 2.2 billion features per m² (1 feature/441×10⁻¹² m²). In some cases, a 4.5 m² substrate comprise about 10 billion features, each with a 10 um diameter.

In some instances, a deposition device comprises about 2,048 nozzles that each deposit about 100,000 droplets per second at 1 nucleobase per droplet. For each deposition device, at least about 1.75×10¹³ nucleobases are deposited on the substrate per day. In some cases, 100 to 500 nucleobase oligonucleic acids are synthesized. In some cases, 200 nucleobase oligonucleic acids are synthesized. Optionally, over 3 days, at a rate of about 1.75×10¹³ bases per day, at least about 262.5×10⁹ oligonucleic acids are synthesized.

In one aspect, provided is an automated system for use with an oligonucleic acid synthesis method described herein that is capable of processing one or more substrates, comprising: a material deposition device for spraying a microdroplet comprising a reagent on a substrate; a scanning transport for scanning the substrate adjacent to the material deposition device to selectively deposit the microdroplet at specified sites; a flow cell for treating the substrate on which the microdroplet is deposited by exposing the substrate to one or more selected fluids; and an alignment unit for aligning the substrate correctly relative to the material deposition device for deposition. In some instances, the system optionally comprises a treating transport for moving the substrate between the material deposition device and the flow cell for treatment in the flow cell, where the treating transport and said scanning transport are different elements. In other instances, the system does not comprise a treating transport.

In some instances, a device for application of one or more reagents to a substrate during a synthesis reaction is an oligonucleic acid synthesizer comprising a plurality of material deposition devices. In some instances, each material deposition device is configured to deposit nucleotide monomers for phosphoramidite synthesis. In some instances, the oligonucleic acid synthesizer deposits reagents to distinct features of a substrate. Reagents for oligonucleic acid synthesis include reagents for oligonucleic acid extension and wash buffers. As non-limiting examples, the oligonucleic acid synthesizer deposits coupling reagents, capping reagents, oxidizers, de-blocking agents, acetonitrile, gases such as nitrogen gas, and any combination thereof. In addition, the oligonucleic acid synthesizer optionally deposits reagents for the preparation and/or maintenance of substrate integrity. In some instances, the oligonucleic acid synthesizer deposits a drop having a diameter less than about 200 um, 100 um, or 50 um in a volume less than about 1000, 500, 100, 50, or 20 pl. In some cases, the oligonucleic acid synthesizer deposits between about 1 and 10000, 1 and 5000, 100 and 5000, or 1000 and 5000 droplets per second. In some instances, the oligonucleic acid synthesizer uses organic solvents.

In some instances, during oligonucleic acid synthesis, the substrate is positioned within and/or sealed within a flow cell. In some instances, the flow cell provides continuous or discontinuous flow of liquids such as those comprising reagents necessary for reactions within the substrate, for example, oxidizers and/or solvents. In some instances, the flow cell provides continuous or discontinuous flow of a gas, such as nitrogen, for drying the substrate typically through enhanced evaporation of a volatile substrate. A variety of auxiliary devices are useful to improve drying and reduce residual moisture on the surface of the substrate. Examples of such auxiliary drying devices include, without limitation, a vacuum source, depressurizing pump and a vacuum tank. In some cases, an oligonucleic acid synthesis system comprises one or more flow cells, such as 2, 3, 4, 5, 6, 7, 8, 9, 10, or 20 and one or more substrates, such as 2, 3, 4, 5, 6, 7, 8, 9, 10 or 20. In some cases, a flow cell is configured to hold and provide reagents to the substrate during one or more steps in a synthesis reaction. In some instances, a flowcell comprises a lid that slides over the top of a substrate and can be clamped into place to form a pressure tight seal around the edge of the substrate. An adequate seal includes, without limitation, a seal that allows for about 1, 2, 3, 4, 5, 6, 7, 8, 9 or 10 atmospheres of pressure. In some cases, the lid of the flow cell is opened to allow for access to an application device such as an oligonucleic acid synthesizer. In some cases, one or more steps of an oligonucleic acid synthesis method are performed on a substrate within a flow cell, without the transport of the substrate.

In some instances, a device for treating a substrate with a fluid comprises a spray bar. In an exemplary oligonucleic acid synthesis process, nucleotide monomers are applied onto a substrate surface with an application device and then a spray bar sprays the substrate surface with one or more treatment reagents using spray nozzles of the spray bar. In some instances, the spray nozzles are sequentially ordered to correlate with different treatment steps during oligonucleic acid synthesis. The chemicals used in different process steps are easily changed in the spray bar to readily accommodate changes in a synthesis method or between steps of a synthesis method. In some instances, the spray bar continuously sprays a given chemistry on a surface of a substrate as the substrate moves past the spray bar. In some cases, the spray bar deposits over a wide area of a substrate, much like the spray bars used in lawn sprinklers. In some instances, the spray bar nozzles are positioned to provide a uniform coat of treatment material to a given area of a substrate.

In some instances, an oligonucleic acid synthesis system comprises one or more elements useful for downstream processing of synthesized oligonucleic acids. As an example, the system comprises a temperature control element such as a thermal cycling device. In some instances, the temperature control element is used with a plurality of resolved reactors to perform nucleic acid assembly such as PCA and/or nucleic acid amplification such as PCR.

The oligonucleic acid synthesizer includes a material deposition device that moves in the X-Y direction to align with the location of the substrate. The oligonucleic acid synthesizer can also move in the Z direction to seal with the substrate, forming a resolved reactor. A resolved reactor is configured to allow for the transfer of fluid, including oligonucleic acids and/or reagents, from the substrate to a capping element and/or vice versa. Fluid may pass through either or both the substrate and the capping element and includes, without limitation, coupling reagents, capping reagents, oxidizers, de-blocking agents, acetonitrile and nitrogen gas.

An oligonucleic acid synthesizer comprises one or more deposition devices that deposit reagents for nucleic acid synthesis onto distinct features or regions of a substrate at a high resolution. Examples of devices that are capable of high resolution droplet deposition include the printhead of inkjet printers and laser printers. The devices useful in the systems and methods described herein achieve a resolution from about 100 dots per inch (DPI) to about 50,000 DPI; from about 100 DPI to about 20,000 DPI; from about 100 DPI to about 10,000 DPI; from about 100 DPI to about 5,000 DPI; from about 1,000 DPI to about 20,000 DPI; or from about 1,000 DPI to about 10,000 DPI. In some cases, the devices have a resolution at least about 1,000; 2,000; 3,000; 4,000; 5,000; 10,000; or 20,000 DPI. The high resolution deposition performed by the device is related to the number and density of each nozzle that corresponds to a feature of the substrate.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some cases, the droplet size is less than about 1 pl, 0.5 pl, 0.2 pl, 0.1 pl, or 0.05 pl. The size of droplets dispensed by the device is correlated to the diameters of deposition nozzles, wherein each nozzle is capable of depositing a reagent onto a feature of the substrate. In some instances, a deposition device of an oligonucleic acid synthesizer comprises from about 100 to about 10,000 nozzles; from about 100 to about 5,000 nozzles; from about 100 to about 3,000 nozzles; from about 500 to about 10,000 nozzles; or from about 100 to about 5,000 nozzles. In some cases, the deposition device comprises greater than 1,000; 2,000; 3,000; 4,000; 5,000; or 10,000 nozzles. In some cases, each material deposition device comprises a plurality of nozzles, where each nozzle is optionally configured to correspond to a feature on a substrate. In some cases, each nozzle deposits a reagent component that is different from another nozzle. In some instances, each nozzle deposits a droplet that covers one or more features of the substrate. In some instances, one or more nozzles are angled. In some instances, multiple deposition devices are stacked side by side to achieve a fold increase in throughput. In some cases, the gain is 2×, 4×, 8× or more. An example of a deposition device is Samba Printhead (Fujifilm). A Samba Printhead may be used with the Samba Web Administration Tool (SWAT).

In some oligonucleic acid synthesis methods, nucleic acid reagents are deposited on the substrate surface in a non-continuous, or drop-on-demand method. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some cases, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some cases, the frequency is less than about 500 KHz, 200 KHz, 100 KHz, or 50 KHz.

In some instances, the number of deposition sites increases by using and rotating the same deposition device by a certain degree or saber angle. By rotating the deposition device, each nozzle is jetted with a certain amount of delay time corresponding to the saber angle. This unsynchronized jetting creates a cross talk among the nozzles. Therefore, when the droplets are jetting at a certain saber angle different from 0 degrees, the droplet volume from the nozzle could be different.

In some instances, the configuration of an oligonucleic acid synthesis system allows for a continuous oligonucleic acid synthesis process that exploits the flexibility of a substrate for traveling in a reel-to-reel type process. This synthesis process operates in a continuous production line manner with the substrate travelling through various stages of oligonucleic acid synthesis using one or more reels to rotate the position of the substrate. In some instances, an oligonucleic acid synthesis reaction comprises rolling a substrate: through a solvent bath, beneath a deposition device for phosphoramidite deposition, through a bath of oxidizing agent, through an acetonitrile wash bath, and through a deblock bath. Optionally, the tape is also traversed through a capping bath. A reel-to-reel type process allows for the finished product of a substrate comprising synthesized oligonucleic acids to be easily gathered on a take-up reel, where it can be transported for further processing or storage.

In some instances, oligonucleic acid synthesis proceeds in a continuous process as a continuous flexible tape is conveyed along a conveyor belt system. Similar to the reel-to-reel type process, oligonucleic acid synthesis on a continuous tape operates in a production line manner, with the substrate travelling through various stages of oligonucleic acid synthesis during conveyance. However, in a conveyor belt process, the continuous tape revisits an oligonucleic acid synthesis step without rolling and unrolling of the tape, as in a reel-to-reel process. In some instances, oligonucleic acid synthesis steps are partitioned into zones and a continuous tape is conveyed through each zone one or more times in a cycle. In some instances, an oligonucleic acid synthesis reaction comprises (1) conveying a substrate through a solvent bath, beneath a deposition device for phosphoramidite deposition, through a bath of oxidizing agent, through an acetonitrile wash bath, and through a block bath in a cycle; and then (2) repeating the cycle as necessary to achieve synthesized oligonucleic acids of a predetermined length. In some cases, after oligonucleic acid synthesis, the flexible substrate is removed from the conveyor belt system and rolled, optionally around a reel, for storage.

### Computer Systems

In various aspects, any of the systems described herein are operably linked to a computer and are optionally automated through a computer either locally or remotely. In some instances, the methods and systems described herein further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate.

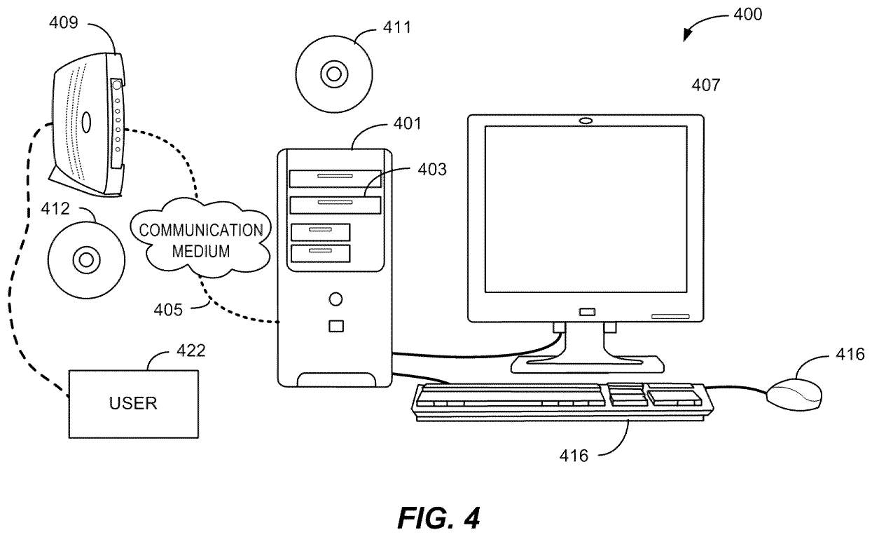

The computer system 400 illustrated in FIG. 4 may be understood as a logical apparatus that can read instructions from media 411 and/or a network port 405, which can optionally be connected to server 409 having fixed media 412. The system, such as shown in FIG. 4 can include a CPU 401, disk drives 403, optional input devices such as keyboard 415 and/or mouse 416 and optional monitor 407. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 422 as illustrated in FIG. 4.

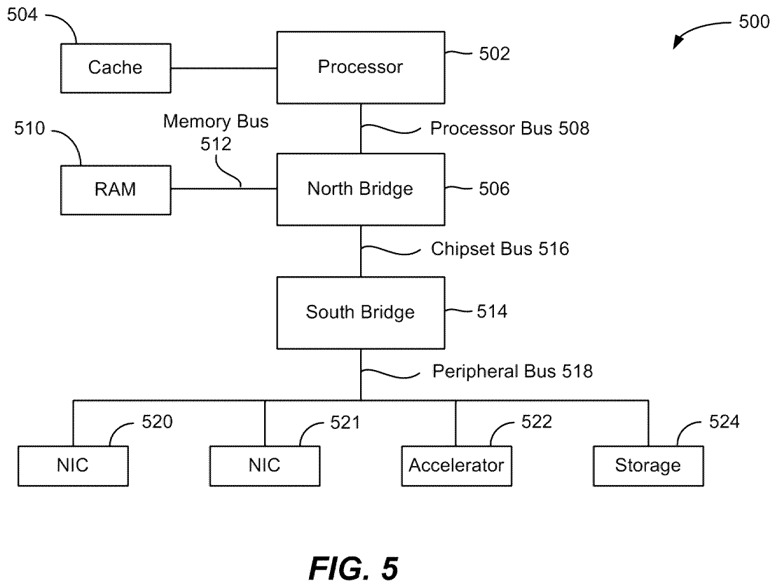

FIG. 5 is a block diagram illustrating a first example architecture of a computer system 500 that can be used in connection with example embodiments of the present invention. As depicted in FIG. 5, the example computer system can include a processor 502 for processing instructions. Non-limiting examples of processors include: Intel Xeon™ processor, AMD Opteron™ processor, Samsung 32-bit RISC ARM 1176JZ (F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some embodiments, multiple processors or processors with multiple cores can also be used, whether in a single computer system, in a cluster, or distributed across systems over a network comprising a plurality of computers, cell phones, and/or personal data assistant devices.

As illustrated in FIG. 5, a high speed cache 504 can be connected to, or incorporated in, the processor 502 to provide a high speed memory for instructions or data that have been recently, or are frequently, used by processor 502. The processor 502 is connected to a north bridge 506 by a processor bus 508. The north bridge 506 is connected to random access memory (RAM) 510 by a memory bus 512 and manages access to the RAM 510 by the processor 502. The north bridge 506 is also connected to a south bridge 514 by a chipset bus 516. The south bridge 514 is, in turn, connected to a peripheral bus 518. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 518. In some alternative architectures, the functionality of the north bridge can be incorporated into the processor instead of using a separate north bridge chip.

In some embodiments, system 500 can include an accelerator card 522 attached to the peripheral bus 518. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 524 and can be loaded into RAM 510 and/or cache 504 for use by the processor. The system 500 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention.

In this example, system 500 also includes network interface cards (NICs) 520 and 521 connected to the peripheral bus for providing network interfaces to external storage, such as Network Attached Storage (NAS) and other computer systems that can be used for distributed parallel processing.

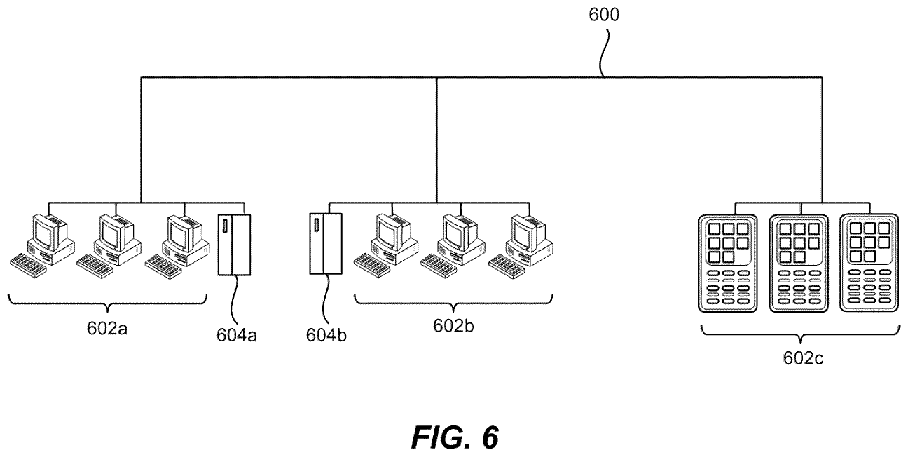

FIG. 6 is a diagram showing a network 600 with a plurality of computer systems 602a, and 602b, a plurality of cell phones and personal data assistants 602c, and Network Attached Storage (NAS) 604a, and 604b. In example embodiments, systems 602a, 602b, and 602c can manage data storage and optimize data access for data stored in Network Attached Storage (NAS) 604a and 604b. A mathematical model can be used for the data and be evaluated using distributed parallel processing across computer systems 602a, and 602b, and cell phone and personal data assistant systems 602c. Computer systems 602a, and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

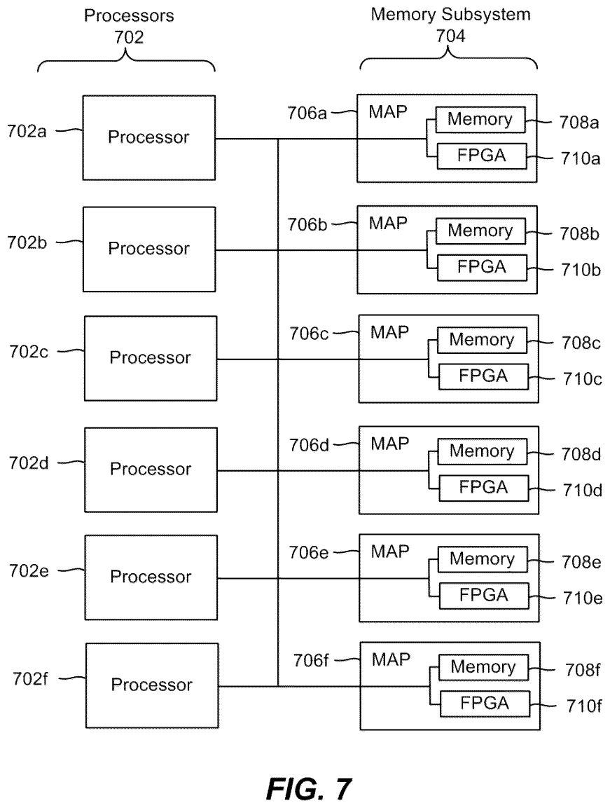

FIG. 7 is a block diagram of a multiprocessor computer system 700 using a shared virtual address memory space in accordance with an example embodiment. The system includes a plurality of processors 702a-f that can access a shared memory subsystem 704. The system incorporates a plurality of programmable hardware memory algorithm processors (MAPs) 706a-f in the memory subsystem 704. Each MAP 706a-f can comprise a memory 708a-f and one or more field programmable gate arrays (FPGAs) 710a-f. The MAP provides a configurable functional unit and particular algorithms or portions of algorithms can be provided to the FPGAs 710a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 708a-f, allowing it to execute tasks independently of, and asynchronously from, the respective microprocessor 702a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs) as referenced in FIG. 7, system on chips (SOCs), application specific integrated circuits (ASICs), or other processing and logic elements. For example, the Set Processor and Optimizer can be implemented with hardware acceleration through the use of a hardware accelerator card.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Functionalization of a Device Surface

A device was functionalized to support the attachment and synthesis of a library of oligonucleic acids. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N2. The device was subsequently soaked in NH₄OH (1:100; 3 mL: 300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N2. The functionalized surface was activated to serve as a support for oligonucleic acid synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary oligonucleotide of 50 bp (“50-mer oligonucleotide”) using oligonucleotide synthesis methods described herein.

The sequence of the 50-mer was as described in SEQ ID NO.: 1. 5′AGACAATCAACCATTTGGGGTGGACAGCCTTGACCTCTAGACTTCGGCAT##TTTTTTT TTT3′ (SEQ ID NO.: 1), where #denotes Thymidine-succinyl hexamide CED phosphoramidite (CLP-2244 from ChemGenes), which is a cleavable linker enabling the release of oligonucleic acids from the surface during deprotection.

The synthesis was done using standard DNA synthesis chemistry (coupling, capping, oxidation, and deblocking) according to the protocol in Table 1 and an ABI synthesizer.

TABLE 1

| General DNA Synthesis | | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I2 in 20% pyridine, 10% water, and 70% THF) were roughly ˜100 μL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 μL/sec, and for Deblock (3% dichloroacetic acid in toluene), roughly ˜300 μL/sec (compared to ˜50 μL/sec for all reagents with flow restrictor). The time to completely push out Oxidizer was observed, the timing for chemical flow times was adjusted accordingly and an extra ACN wash was introduced between different chemicals. After oligonucleotide synthesis, the chip was deprotected in gaseous ammonia overnight at 75 psi. Five drops of water were applied to the surface to recover oligonucleic acids. The recovered oligonucleic acids were then analyzed on a BioAnalyzer small RNA chip (data not shown).

### Example 3: Synthesis of a 100-Mer Sequence on an Oligonucleotide Synthesis Device

The same process as described in Example 2 for the synthesis of the 50-mer sequence was used for the synthesis of a 100-mer oligonucleotide (“100-mer oligonucleotide”; 5′ CGGGATCCTTATCGTCATCGTCGTACAGATCCCGACCCATTTGCTGTCCACCAGTCATGC TAGCCATACCATGATGATGATGATGATGAGAACCCCGCAT##TTTTTTTTTT3′, where #denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the oligonucleic acids extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 mastermix, 2.5 uL 10 uM Forward primer, 2.5 uL 10 uM Reverse primer, 1 uL oligonucleic acid extracted from the surface, and water up to 50 uL) using the following thermalcycling program:
98 C, 30 sec
98 C, 10 sec; 63 C, 10 sec; 72 C, 10 sec; repeat 12 cycles
72 C, 2 min

The PCR products were also run on a BioAnalyzer (data not shown), demonstrating sharp peaks at the 100-mer position. Next, the PCR amplified samples were cloned, and Sanger sequenced. Table 2 summarizes the results from the Sanger sequencing for samples taken from spots 1-5 from chip 1 and for samples taken from spots 6-10 from chip 2.

TABLE 2

| | | |
|---|---|---|
| Spot | Error rate | Cycle efficiency |

| | | | |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized oligonucleotides were repeated on two chips with different surface chemistries. Overall, 89%, corresponding to 233 out of 262 of the 100-mers that were sequenced were perfect sequences with no errors.

Finally, Table 3 summarizes error characteristics for the sequences obtained from the oligonucleotides samples from spots 1-10.

TABLE 3

| Sample | | | | | | |
|---|---|---|---|---|---|---|
| ID/Spot | | | | | | |
| no. | OSA_0046/1 | OSA_0047/2 | OSA_0048/3 | OSA_0049/4 | OSA_0050/5 | OSA_0051/6 |
| Total | 32 | 32 | 32 | 32 | 32 | 32 |
| Sequences | | | | | | |
| Sequencing | 25 of | 27 of | 26 of | 21 of | 25 of | 29 of |
| Quality | 28 | 27 | 30 | 23 | 26 | 30 |
| Oligo | 23 of | 25 of | 22 of | 18 of | 24 of | 25 of |
| Quality | 25 | 27 | 26 | 21 | 25 | 29 |
| ROI Match | 2500 | 2698 | 2561 | 2122 | 2499 | 2666 |
| Count | | | | | | |
| ROI | 2 | 2 | 1 | 3 | 1 | 0 |
| Mutation | | | | | | |
| ROI Multi | 0 | 0 | 0 | 0 | 0 | 0 |
| Base Deletion | | | | | | |
| ROI Small | 1 | 0 | 0 | 0 | 0 | 0 |
| Insertion | | | | | | |
| ROI Single | 0 | 0 | 0 | 0 | 0 | 0 |
| Base Deletion | | | | | | |
| Large Deletion | 0 | 0 | 1 | 0 | 0 | 1 |
| Count | | | | | | |
| Mutation: | 2 | 2 | 1 | 2 | 1 | 0 |
| G > A | | | | | | |
| Mutation: | 0 | 0 | 0 | 1 | 0 | 0 |
| T > C | | | | | | |
| ROI Error | 3 | 2 | 2 | 3 | 1 | 1 |
| Count | | | | | | |
| ROI Error | Err: ~1 | Err: ~1 | Err: ~1 | Err: ~1 | Err: ~1 | Err: ~1 |
| Rate | in 834 | in 1350 | in 1282 | in 708 | in 2500 | in 2667 |
| ROI Minus | MP Err: ~1 | MP Err: ~1 | MP Err: ~1 | MP Err: ~1 | MP Err: ~1 | MP Err: ~1 |
| Primer | in 763 | in 824 | in 780 | in 429 | in 1525 | in 1615 |
| Error Rate | | | | | | |

| Sample | | | | |
|---|---|---|---|---|
| ID/Spot | | | | |
| no. | OSA_0052/7 | OSA_0053/8 | OSA_0054/9 | OSA_0055/10 |
| Total | 32 | 32 | 32 | 32 |
| Sequences | | | | |
| Sequencing | 27 of | 29 of | 28 of | 25 of |
| Quality | 31 | 31 | 29 | 28 |
| Oligo | 22 of | 28 of | 26 of | 20 of |
| Quality | 27 | 29 | 28 | 25 |
| ROI Match | 2625 | 2899 | 2798 | 2348 |
| Count | | | | |
| ROI | 2 | 1 | 2 | 1 |
| Mutation | | | | |
| ROI Multi | 0 | 0 | 0 | 0 |
| Base Deletion | | | | |
| ROI Small | 0 | 0 | 0 | 0 |
| Insertion | | | | |
| ROI Single | 0 | 0 | 0 | 0 |
| Base Deletion | | | | |
| Large Deletion | 1 | 0 | 0 | 0 |
| Count | | | | |
| Mutation: | 2 | 1 | 2 | 1 |
| G > A | | | | |
| Mutation: | 0 | 0 | 0 | 0 |
| T > C | | | | |
| ROI Error | 3 | 1 | 2 | 1 |
| Count | | | | |
| ROI Error | Err: ~1 | Err: ~1 | Err: ~1 | Err: ~1 |
| Rate | in 876 | in 2900 | in 1400 | in 2349 |
| ROI Minus | MP Err: ~1 | MP Err: ~1 | MP Err: ~1 | MP Err: ~1 |
| Primer | in 531 | in 1769 | in 854 | in 1451 |
| Error Rate | | | | |

### Example 4: Highly Accurate DNA-Based Information Storage and Recovery

Digital information was selected in the form of binary data totaling about 0.2 GB included content for the Universal Declaration of Human Rights in more than 100 languages, the top 100 books of Project Guttenberg and a seed database. The digital information was encrypted into a nucleic acid-based sequence and divided into strings. Over 10 million non-identical oligonucleic acids, each corresponding to a string, were synthesized on a rigid silicon surface in a manner similar to that described in Example 2. Each non-identical oligonucleic acid was under equal or less than 200 bases in length. The synthesized oligonucleic acids were collected and sequenced and decoded back to digital code, with 100% accuracy for the source digital information.

### Example 5: Flexible Substrate Having a High Density of Features

A flexible structure comprising thermoplastic material is coated with a nucleoside coupling reagent. The coating agent is patterned for a high density of features. A portion of the flexible surface is illustrated in FIG. 2B. Each feature has a diameter of 10 um, with a center-to-center distance between two adjacent features of 21 μm. The feature size is sufficient to accommodate a sessile drop volume of 0.2 pl during an oligonucleic acid synthesis deposition step. The small feature dimensions allow for a high density of oligonucleic acids to be synthesized on the surface of the substrate. The feature density is 2.2 billion features/m² (1 feature/441×10⁻¹² m²). A 4.5 m² substrate is manufactured having 10 billion features, each with a 10 um diameter. The flexible structure is optionally placed in a continuous loop system, FIG. 2A, for oligonucleic acid synthesis.

### Example 6: Oligonucleic Acid Synthesis on a Flexible Substrate

A flexible substrate is prepared comprising a plurality of features on a thermoplastic flexible material. The substrate serves as a support for the synthesis of oligonucleic acids using an oligonucleic acid synthesis device comprising a deposition device. The flexible substrate is in the form of a flexible media much like a magnetic reel-to-reel tape.

De novo synthesis operates in a continuous production line manner with the substrate travelling through a solvent bath and then beneath a stack of printheads where the phosphoramidites are printed on to the substrate. The flexible substrate with the sessile drops deposited on to the surface is rolled into a bath of oxidizing agent, then the tape emerges from the oxidizing bath and is immersed in an acetonitrile wash bath then submerged in a deblock bath. Optionally, the tape is traversed through a capping bath. In an alternative workflow, the flexible substrate emerges from the oxidizing bath and is sprayed with acetonitrile in a wash step.

Alternatively, a spray bar is used instead of a liquid bath. In this process, the nucleotides are still deposited on the surface with an inkjet device but the flood steps are now done in a chamber with a spray nozzles. For example, the deposition device has 2,048 nozzles that each deposit 100,000 droplets per second at 1 nucleobase per droplet. There is a sequential ordering of spray nozzles to mimic the ordering of the flood steps in standard phosphoramidite chemistry. This technique provides for easily changing the chemicals loaded in the spray bar to accommodate different process steps. Oligonucleic acids are deprotected or cleaved in the same manner as described in Example 2.

For each deposition device, more than 1.75×10¹³ nucleobases are deposited on the substrate per day (24 hours). A plurality of 200 nucleobase oligonucleic acids is synthesized. In 3 days (72 hours), at a rate of 1.75×10¹³ bases per day, 262.5×10⁹ oligonucleic acids are synthesized.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-16. (canceled)

17. A method for storing information, the method comprising:
a) converting an item of information in the form of at least one digital sequence to at least one nucleic acid sequence;
b) synthesizing a plurality of oligonucleic acids having predetermined sequences collectively encoding for the at least one nucleic acid sequence, wherein the plurality of oligonucleic acids comprises at least about 10,000 oligonucleic acids, wherein the plurality of oligonucleic acids collectively encode for a sequence that differs from the predetermined sequences by no more than 1 base in 1000, and wherein each oligonucleic acid of the plurality of oligonucleic acids comprises from 50 to 500 bases in length; and
c) storing the plurality of oligonucleic acids.

18-21. (canceled)

22. The method of claim 17, wherein the item of information is text information, audio information or visual information.

23. The method of claim 17, further comprising providing a structure, wherein the structure is rigid or flexible, and wherein the structure comprises a surface, and wherein the plurality of oligonucleic acids extend from the surface.

24-29. (canceled)

30. A method for storing information, the method comprising:
a) encrypting at least one item of information in the form of at least one digital sequence to at least one nucleic acid sequence;
b) synthesizing a plurality of oligonucleic acids having predetermined sequences collectively encoding for the at least one nucleic acid sequence, wherein the plurality of oligonucleic acids comprises at least about 10,000 oligonucleic acids, and wherein each oligonucleic acid of the plurality of oligonucleic acids comprises from 50 to 500 bases in length;
c) storing the plurality of oligonucleic acids;
d) sequencing the plurality of oligonucleic acids;
e) decrypting the plurality of oligonucleic acids from a nucleic acid sequence to a digital sequence; and
f) assembling the digital sequence to form the at least one digital sequence, wherein the at least one digital sequence is recovered with 100% accuracy.

31. A device for information storage, the device comprising:
a) a flexible structure having a surface; and
b) a plurality of features on the surface, wherein each feature has a width of from about 1 to about 500 μm, and wherein each feature of the plurality of features is coated with a moiety that binds to the surface and comprises a hydroxyl group available for nucleoside coupling.

32. The device of claim 31, wherein the flexible structure rests in a curved position.

33. The device of claim 32, wherein the curved position comprises a curve that is greater than 30 degrees.

34. The device of claim 31, wherein the flexible structure comprises at least about 1 million features.

35. The device of claim 31, wherein the flexible structure has a total surface area of less than about 4.5 m².

36. The device of claim 31, wherein the flexible structure comprises more than 2 billion features per m².

37. The device of claim 31, wherein the flexible structure comprises a thermoplastic material.

38. The device of claim 37, wherein the thermoplastic material comprises a polyaryletherketone.

39. The device of claim 38, wherein the polyaryletherketone is polyetherketone, polyetherketoneketone, poly(ether ether ketone ketone), polyether ether ketone or polyetherketoneetherketoneketone.

40. The device of claim 31, wherein the flexible structure comprises nylon, nitrocellulose, polypropylene, polycarbonate, polyethylene, polyurethane, polystyrene, acetal, acrylic, acrylonitrile, butadiene styrene, polyethylene terephthalate, polymethyl methacrylate, polyvinyl chloride, transparent PVC foil, Poly(methyl methacrylate), styrenic polymer, fluorine-containing polymers, polyethersulfone or polyimide.

41. The device of claim 31, wherein the flexible structure has a thickness of less than about 10 mm.

42. The device of claim 31, wherein each feature is from about 1 um to about 50 um in width.

43. The device of claim 31, wherein each feature has a diameter of about 10 um.

44. The device of claim 31, wherein the flexible structure comprises a reel-to-reel tape or a continuous tape.

45. The device of claim 31, wherein each feature comprises a channel.
